(19) United States
(12) Patent Application Publication
(10) Pub. No.: US 2022/0168312 A1
(43) Pub. Date: Jun. 2, 2022

(54) MULTICOMPONENT CRYSTAL FORMULATIONS

(71) Applicant: UNIVERSITY OF LIMERICK, Limerick (IE)

(72) Inventors: Oisín KAVANAGH, Limerick (IE); Matteo LUSI, Limerick (IE); Gavin WALKER, Limerick (IE)

(21) Appl. No.: 17/600,860

(22) PCT Filed: Apr. 2, 2020
(86) PCT No.: PCT/EP2020/059463

§ 371 Date: Oct. 1, 2021

(30) Foreign Application Priority Data
Apr. 2, 2019 (EP) 19166734.4

(51) Int. Cl. A61K 31/53; A61K 31/192; A61K 31/431; A61K 31/43; A61K 31/545; A61K 31/546; A61K 31/606; A61K 31/496; A61K 31/4995; A61K 9/14

(52) U.S. Cl. CPC A61K 31/53; A61K 31/192; A61K 31/431; A61K 31/43; A61K 9/14; A61K 31/546; A61K 31/606; A61K 31/496; A61K 31/4995; A61K 31/545

(57) ABSTRACT

A multicomponent crystal (or co-crystal) comprising a first active pharmaceutical ingredient and a second active pharmaceutical ingredient. The multicomponent crystal is formed/sustained by non-covalent interactions between the nitrogen-containing heterocycle alpha-substituted with an amino group of the first active pharmaceutical ingredient and a carboxylic acid group of the second active pharmaceutical ingredient, suitably as well as other further non-covalent interactions with other H-bond forming groups. The multicomponent crystal may provide an improved multidrug dosage form comprising lamotrigine and valproic acid as the first and second active pharmaceutical ingredients, respectively. A pharmaceutical composition comprising a therapeutically effective amount of the multicomponent crystal and a pharmaceutically acceptable excipient, and a method of forming the multicomponent crystal, are also provided.

### FIELD

The present invention relates to a multicomponent crystal (or co-crystal) comprising a first active pharmaceutical ingredient and a second active pharmaceutical ingredient, and to a pharmaceutical composition comprising a therapeutically effective amount of such a multicomponent crystal. In particular, the present invention relates to a multicomponent crystal comprising at least an interaction between a nitrogen-containing heterocycle substituted with an amino group of the first active pharmaceutical ingredient and a carboxylate group of the second active pharmaceutical ingredient, suitably further supported by additional H-bond interactions.

### BACKGROUND

Many complex medical conditions require treatment with a combination of drugs, with each targeting a different aspect of the disease. However, complex drug regimens negatively affect patience compliance and result in poor adherence to therapy, which can lead to serious health consequences and increased costs to healthcare systems. In epilepsy, for instance, a single missed dose can lead to a fatal seizure. By enabling simpler drug regimens, multidrug dosage forms could provide safer and more effective therapeutic alternatives.

A multidrug dosage form can be obtained by co-formulating multiple active pharmaceutical ingredients (APIs) in the appropriate ratio to deliver the required effective amount of each API to the patient. However, only chemically compatible drug substances can be formulated together and the addition of excipients might be required to ensure adequate stability and bioavailability.

Therefore there is a need for multidrug dosage forms which have adequate or improved physical, pharmacokinetic and pharmacodynamic properties.

In epilepsy, adequate seizure control often requires combination therapy of lamotrigine and valproic acid. Lamotrigine is a solid class II drug (highly permeable and poorly soluble) according to the biopharmaceutical classification system (BCS). Orally it is 90% bioavailable but has a rather short half-life. Valproic acid is a liquid, which is predominantly marketed in its ionic co-crystal form as Depakote® or Epilim® (valproic acid and sodium valproate). In clinical use, the drug dosage is titrated according to seizure control. Therefore a multidrug dosage form comprising lamotrigine and valproic acid having good physical, pharmacokinetic and pharmacodynamic properties would be beneficial.

Co-crystals are solids that have a single crystalline single phase composed of two or more different molecular and/or ionic compounds. When one or both of the compounds in the co-crystal are ionic (i.e. salts), the resulting co-crystal is an ionic co-crystal. When both of the compounds of the co-crystal are neutral or zwitterionic molecules, the resulting co-crystal is a molecular co-crystal. A pharmaceutical co-crystal may be defined as a co-crystal which comprises an active pharmaceutical ingredient and a pharmaceutically acceptable compound (e.g. an excipient). The term “multicomponent crystal” may be used herein instead of “co-crystal” wherein at least one of the components of the “co-crystal” is in an ionic form.

### SUMMARY OF THE INVENTION

It is one aim of the present invention, amongst others, to provide a multidrug dosage form that addresses at least one disadvantage of the prior art, whether identified here or elsewhere, or to provide an alternative to existing multidrug dosage forms. For instance it may be an aim of the present invention to provide a multicomponent crystal of a first API and a second API which may have improved drug properties compared to either of the first or second APIs alone.

According to aspects of the present invention, there is provided a multicomponent crystal and a pharmaceutical composition comprising a therapeutically effective amount and ratio of such components in a multicomponent crystal, as set forth in the appended claims. Other features of the invention will be apparent from the dependent claims, and the description which follows.

According to a first aspect of the present invention, there is provided a multicomponent crystal of a first active pharmaceutical ingredient and a second active pharmaceutical ingredient;

wherein the first active pharmaceutical ingredient comprises a nitrogen-containing heterocycle substituted with an amino group;

wherein the second active pharmaceutical ingredient comprises a carboxylic acid; and

wherein the nitrogen-containing heterocycle substituted with an amino group of the first active pharmaceutical ingredient interacts with the carboxylic acid of the second active pharmaceutical ingredient, in the multicomponent crystal.

The reference to the first and second active pharmaceutical ingredients is intended to mean that the first and second active pharmaceutical ingredients are different, i.e. different chemical entities, suitably having different pharmaceutical effects.

In the multicomponent crystal of this first aspect, the nitrogen-containing heterocycle substituted with an amino group of the first active pharmaceutical ingredient interacts with the carboxylic acid of the second active pharmaceutical ingredient. This interaction is suitably a non-covalent interaction between the stated functional groups, suitably a hydrogen bonding interaction. Suitably this interaction at least partially forms and sustains the multicomponent crystal structure.

Suitably the multicomponent crystal comprises further non-covalent interactions between the between the first and second active pharmaceutical ingredients, suitably further hydrogen bonding interactions. Such additional or secondary interactions suitably also form and sustain the multicomponent crystal structure. Therefore the first active pharmaceutical ingredient and/or the second active pharmaceutical ingredient suitably form non-covalent interactions with other H-bond forming groups in the components of the multicomponent crystal, suitably H-bond interactions. Such other H-bond forming groups may be present in the first and/or second active pharmaceutical ingredients, or may be part of a third or further component in the multicomponent crystal, as discussed below.

Suitably the nitrogen-containing heterocycle of the first active pharmaceutical ingredient is alpha-substituted with the amino group. Therefore the amino group is suitably attached to a carbon atom adjacent to a nitrogen atom in the nitrogen-containing heterocycle.

The first and second active pharmaceutical ingredients may be present in the multicomponent crystal either in neutral or ionised form (i.e. the first active pharmaceutical ingredient may be protonated and the second active pharmaceutical ingredient may be deprotonated). The multicomponent crystal may comprise either or both of the neutral or ionised forms of either or both of the first and second active pharmaceutical ingredients.

The multicomponent crystal of this first aspect may be alternatively or additionally defined as a co-crystal of the stated components, insofar as that term can be used to cover such crystals comprising at least some ionic forms of the stated components.

The multicomponent crystal of this first aspect may be considered to be a crystalline composition comprising the stated multicomponent crystal form, suitably consisting essentially or, or consisting of, the stated multicomponent crystal form. The multicomponent crystal of this first aspect comprises a first API and a second API and therefore may be considered to be a multidrug dosage form consisting of such a multicomponent crystal. The multicomponent crystal of this first aspect may be additionally or alternatively referred to as a combination active pharmaceutical ingredient, which may be further formulated with suitable pharmaceutically acceptable excipients to provide a pharmaceutical composition for use as a medicament.

The inventors have surprisingly found that APIs having the stated functional groups can advantageously form a multicomponent crystal, comprising ionised components which, when sustained by H-bond interactions, may provide advantageous drug properties such as improved solubility, tablet-ability, thermal stability and chemical stability.

In some embodiments the second API may have a different, reduced or minor pharmaceutical effect compared to the first API. The second API may therefore be intended to treat a different condition compared to the first API. The second API may have some biological activity but may be present in the multicomponent crystal of this first aspect primarily to improve the drug properties of the multicomponent crystal, such as any of the properties referred to above, rather than to provide pharmaceutical activity. For example, the second API may be a pharmaceutically acceptable excipient with little or no biological activity.

In such embodiments, the multicomponent crystal of this first aspect can be considered to comprise a first pharmaceutical ingredient and a second pharmaceutical ingredient;

wherein the first pharmaceutical ingredient is an active pharmaceutical ingredient;

wherein the first pharmaceutical ingredient comprises a nitrogen-containing heterocycle substituted with an amino group;

wherein the second pharmaceutical ingredient comprises a carboxylic acid; and wherein the nitrogen-containing heterocycle substituted with an amino group of the first pharmaceutical ingredient interacts with the carboxylic acid of the second pharmaceutical ingredient, in the multicomponent crystal.

In such embodiments, the second pharmaceutical ingredient may be a carboxylic compound, for example a carboxylic acid selected from citric, tartaric, acetic, lactic, pyruvic, acetic, trifluoroacetic, succinic, fumaric, maleic, glycolic, lactic, salicylic, oxaloacetic, formic, benzoic, malonic, ascorbic, malic, phthalic, aspartic, and glutamic acids, lysine and arginine. In one embodiment, the second pharmaceutical ingredient is suitably benzoic acid.

In such embodiments, the multicomponent crystal of this first aspect may be considered to be a single drug dosage form consisting of such a multicomponent crystal.

In the following description, references to the first active pharmaceutical ingredient and to the second pharmaceutical ingredient are intended to also apply to the first pharmaceutical ingredient and the second pharmaceutical ingredient, where appropriate, of this embodiment, particularly wherein the second pharmaceutical ingredient has little or no biological activity.

The multicomponent crystal of this first aspect comprises the first and second APIs. Suitably the multicomponent crystal consists essentially or consists of the first and second APIs.

Suitably the first active pharmaceutical ingredient and the second active pharmaceutical ingredient are organic compounds. Suitably the multicomponent crystal comprises an ionic form or both an ionic and a neutral form of the first active pharmaceutical ingredient and an ionic form or both an ionic and a neutral form of the second active pharmaceutical ingredient.

In the multicomponent crystal of this first aspect, the interaction of the nitrogen-containing heterocycle substituted with an amino group of the first active pharmaceutical ingredient and the carboxylic acid group of the second active pharmaceutical ingredient suitably comprises an R₁² (4) or R₂²(8) motif. In some embodiments, the nitrogen-containing heterocycle of the first active pharmaceutical ingredient is protonated on the nitrogen in the alpha position with respect to the amino group and this proton interacts with the carboxylate group of the second active pharmaceutical ingredient, forming a R₁² (4) motif. Alternatively the nitrogen-containing heterocycle of the first active pharmaceutical ingredient is protonated in the alpha position with respect to the amino group and both protons of the amino group and N-substituted aromatic ring interact with the carboxylate group of the second active pharmaceutical ingredient to form an R₂²(8) motif. In both cases the R₁²(4) and R₂²(8) motif may be supported by further H-bond forming groups interacting with the carboxylate group.

Such further H-bond forming groups may be provided by the first and or second active pharmaceutical ingredients, or by additional molecules of the first and or second active pharmaceutical ingredients in the multicomponent crystal unit cell.

In some embodiments such further H-bond forming groups may be provided by a third component of the multicomponent crystal. The third component may be a third active pharmaceutical ingredient or it may be an inactive pharmaceutically acceptable excipient. The third component may be selected from water, carboxylic acids (acetic acid, NSAIDS), amines (pregablin, gabapentin or benzodiazepines), amides (peptides or caffeine), alcohols (methanol, ethanol, propanol or butanol), phenols (serotonin, adrenaline, vanillin or salicylic acids), mineral acids (hydrochloric, hydrobromic, phosphoric, sulfuric acid), imidazoles (fluconazole, itraconazole or metronidazole) or pyrroles (sunitinib, atorvastatin or Protoporphyrin IX).

Suitably the multicomponent crystal of this first aspect comprises a neutral form of at least one of the first or second active pharmaceutical ingredients, or any third component present. Suitably the neutral form of said compound stabilises the crystal structure, suitably by stabilising the the R₁²(4) and R₂²(8) motif discussed above, suitably by H-bonding.

In some embodiments, the multicomponent crystal comprises an ionic form and a neutral form of the first active pharmaceutical ingredient and an ionic form of the second active pharmaceutical ingredient.

In some embodiments, the multicomponent crystal comprises an ionic form and a neutral form of the second active pharmaceutical ingredient and an ionic form of the first active pharmaceutical ingredient.

In some embodiments, the multicomponent crystal comprises an ionic form and a neutral form of the first active pharmaceutical ingredient and an ionic form and a neutral form of the second active pharmaceutical ingredient; wherein the first active pharmaceutical ingredient and the second active pharmaceutical ingredient are organic compounds.

Suitably the first API is any compound having a nitrogen-containing heterocycle substituted, preferably alpha-substituted, with an amino group which is pharmaceutically active. For example, the first API may be selected from lamotrigine, 4-aminopyridine, cytosine, thymine, 5-fluorocytosine, dihydralazine, endralazine, hydralazine, pipofezine, minaprine, cadralazine or cefozopran.

Suitably the second API is any compound having a carboxylic acid or a carboxylate group which is pharmaceutically active. For example the second API may be selected from valproic acid and/or a valproate salt, NSAIDs—including salicylate derivative NSAIDs, p-amino phenol derivative NSAIDs, propionic acid derivative NSAIDs, acetic acid derivative NSAIDs, enolic acid derivative NSAIDs and fenamic acid derivative NSAIDs—non-selective cyclo-oxygenase (cox) inhibitors, selective cyclooxygenase 1 (cox 1) inhibitors, selective cyclooxygenase 2 (cox 2) inhibitors or an antibiotic such as oxacillin, ampicillin, amoxicillin, cephalexin, cephalotin, cephalosporin, p-amino-salicylic acid, ciprofloxacin, enrofloxacin, difloxacin or danofloxacin.

Suitably the nitrogen-containing heterocycle substituted with an amino group of the first API has the structure (I):

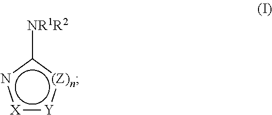

wherein R¹ and R² are each independently selected from H, a C₁-C₈ alkyl, a C₁-C₈ alkenyl, an aryl group, an alkylaryl group, a heteroaryl group or an alkylheteroaryl group, said groups optionally substituted with one or more of C₁-C₄ alkoxy, hydroxy, amino, carboxylic acid, ester, amide, halogen, CF₃, CHF₂ or CH₂F groups;

wherein n=0, 1, 2 or 3;

wherein X, Y and each Z are independently selected from N or C atoms;

wherein said N atoms are optionally substituted with a C₁-C₈ alkyl, a C₁-C₈ alkenyl, an aryl group, an alkylaryl group, a heteroaryl group or an alkylheteroaryl group, said groups optionally substituted with one or more of C₁-C₄ alkoxy, halogen, CF₃, CHF₂ or CH₂F groups; and wherein said C atoms are optionally substituted with C₁-C₈ alkyl, a C₁-C₈ alkenyl, an aryl group, an alkylaryl group, a heteroaryl group or an alkylheteroaryl group, optionally substituted with one or more of C₁-C₄ alkoxy, hydroxy, amino, carboxylic acid, ester, amide, halogen, CF₃, CHF₂ or CH₂F groups, or wherein said C atoms are optionally substituted with NR⁵R⁶, wherein R⁵ and R⁶ are each independently selected from H, a C₁-C₄ alkyl, a C₁-C₄ alkenyl, an aryl group, an alkylaryl group, a heteroaryl group or an alkylheteroaryl group, said groups optionally substituted with one or more of C₁-C₄ alkoxy, hydroxy, amino, carboxylic acid, ester, amide, halogen, CF₃, CHF₂ or CH₂F groups.

Suitably the first API has this structure (I).

Suitably at least one of X, Y and Z is a nitrogen atom. Suitably n=1 or 2.

Suitably at least one of R¹ and R² are H. Suitably both R¹ and R² are H.

Suitably when any of X, Y and each Z are N atoms, said N atoms are unsubstituted, suitably apart from any H atom present to complete the required valency of the N atom in the heterocycle.

Suitably the nitrogen-containing heterocycle substituted with an amino group of the first API has the structure (II):

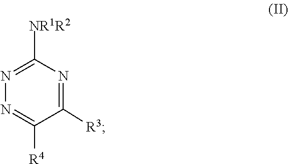

wherein R¹ and R² are each independently selected from H, a C₁-C₈ alkyl, a C₁-C₈ alkenyl, an aryl group, an alkylaryl group, a heteroaryl group or an alkylheteroaryl group, said groups optionally substituted with one or more of C₁-C₄ alkoxy, hydroxy, amino, carboxylic acid, ester, amide, halogen, CF₃, CHF₂ or CH₂F groups; and

wherein R³ and R⁴ are each independently selected from H, NR⁵R⁶, C₁-C₈ alkyl, a C₁-C₈ alkenyl, an aryl group, an alkylaryl group, a heteroaryl group or an alkylheteroaryl group, said groups optionally substituted with one or more of C₁-C₄ alkoxy, hydroxy, amino, carboxylic acid, ester, amide, halogen, CF₃, CHF₂ or CH₂F groups; wherein R⁵ and R⁶ are each independently selected from H, a C₁-C₄ alkyl, a C₁-C₄ alkenyl, an aryl group, an alkylaryl group, a heteroaryl group or an alkylheteroaryl group, said groups optionally substituted with one or more of C₁-C₄ alkoxy, hydroxy, amino, carboxylic acid, ester, amide, halogen, CF₃, CHF₂ or CH₂F groups.

Suitably the nitrogen-containing heterocycle substituted with an amino group of the first API having the structure (I) or (II) comprises a further H-bond forming group, in addition to the amino group. For example, structure (I) or (II) may comprise a further amino group, e.g. NR⁵R⁶ as defined above, a carboxylic acid group, an amide group, an OH group or a suitable heterocyclic group.

Suitably the first API has this structure (II).

Suitably the nitrogen-containing heterocycle substituted with an amino group of the first API has the structure (III):

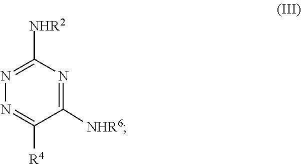

wherein R² is selected from H, a C₁-C₄ alkyl or a C₁-C₄ alkenyl, optionally substituted with one or more of C₁-C₄ alkoxy, hydroxy, amino, halogen, CF₃, CHF₂ or CH₂F groups;

wherein R⁴ is selected from H, C₁-C₈ alkyl, a C₁-C₈ alkenyl, an aryl group, an alkylaryl group, a heteroaryl group or an alkylheteroaryl group, said groups optionally substituted with one or more of C₁-C₄ alkoxy, hydroxy, amino, carboxylic acid, ester, amide, halogen, CF₃, CHF₂ or CH₂F groups; and

wherein R⁶ is selected from H, a C₁-C₄ alkyl, a C₁-C₄ alkenyl, an aryl group, an alkylaryl group, a heteroaryl group or an alkylheteroaryl group, said groups optionally substituted with one or more of C₁-C₄ alkoxy, hydroxy, amino, carboxylic acid, ester, amide, halogen, CF₃, CHF₂ or CH₂F groups.

Suitably the first API has this structure (III).

Suitably R⁶ is selected from H, a C₁-C₄ alkyl or a C₁-C₄ alkenyl, optionally substituted with one or more of C₁-C₄ alkoxy, hydroxy, amino, halogen, CF₃, CHF₂ or CH₂F groups.

Suitably R² and R⁶ are both H.

Suitably R⁴ is an aryl group, optionally substituted with one or more of C₁-C₄ alkoxy, hydroxy, amino, halogen, CF₃, CHF₂ or CH₂F groups.

Suitably the first active pharmaceutical ingredient is lamotrigine. Lamotrigine has the structure (IV):

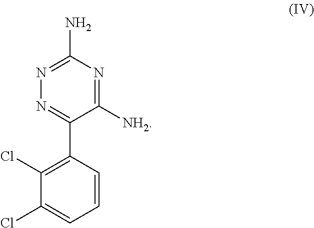 [SMILES: Nc1nnc(-c2cccc(Cl)c2Cl)c(N)n1]

Suitably the second API has the structure (V):

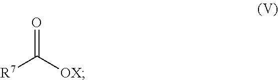

wherein X is H or a negative charge; and

wherein R⁷ is selected from C₁-C₁₀ alkyl, a C₁-C₁₀ alkenyl, an aryl group, an alkylaryl group, a heteroaryl group or an alkylheteroaryl group, optionally substituted with one or more of C₁-C₄ alkoxy, halogen, CF₃, CHF₂ or CH₂F groups.

When X is a negative charge, a suitable positively charged counterion is present in the multicomponent crystal; suitably a protonated and positively charged amino group on the first API.

Suitably R⁷ is selected from C₄-C₁₀ alkyl or a C₄-C₁₀ alkenyl, optionally substituted with one or more of C₁-C₄ alkoxy, hydroxy, amino, carboxylic acid, ester, amide, halogen, CF₃, CHF₂ or CH₂F groups. Suitably R⁷ is a C₄-C₈ alkyl or a C₄-C₈ alkenyl, optionally substituted with one or more of C₁-C₄ alkoxy, halogen, CF₃, CHF₂ or CH₂F groups.

Suitably the second active pharmaceutical ingredient is valproic acid and/or a valproate salt.

Valproic acid has the structure (VI):

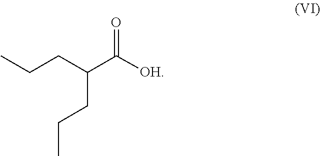

Suitably the first API is lamotrigine and/or a lamotrigine salt and the second API is valproic acid and/or a valproate salt.

The inventors have found that such a multicomponent crystal comprising lamotrigine and valproic acid may provide an advantageous treatment for patients suffering from epilepsy, where lamotrigine and valproic acid are prescribed together. Such a multicomponent crystal may have improved tabletability and solubility compared to know forms of lamotrigine, which are particularly insoluble and difficult to form into tablets (lamotrigine is a solid class II drug—poorly soluble). Furthermore this multicomponent crystal may provide an alternative solid form of valproic acid which is a liquid in its neutral state under ambient conditions, and therefore may provide a more convenient dosage form of valproic acid.

Suitably the molar ratio of the first active pharmaceutical ingredient to the second active pharmaceutical ingredient in the multicomponent crystal is 1:2, suitably wherein the first API is lamotrigine and/or a lamotrigine salt and the second API is valproic acid and/or a valproate salt.

Patients being treated for epilepsy may receive a dual therapy comprising a maintenance dose of 1 g of valproic acid and a 100-200 mg dose of lamotrigine. Therefore a molar ratio of lamotrigine to valproic acid of approximately 1:9. However, due to pharmacodynamic and pharmacokinetic interactions, a 200 mg (0.781 mmol) does of lamotrigine may be used with a lower dose of valproic acid (250 mg, 1.736 mmol). This provides a combined dose having a molar ratio of lamotrigine to valproic acid of approximately 1:2, which prolongs the circulation time of lamotrigine in vivo, and produces a synergistic effect. Therefore the multicomponent crystal of this first aspect comprising a 1:2 ratio of lamotrigine and valproic acid (and/or a valproate salt) may provide a particularly advantageous dosage form of this combined therapy for epilepsy which may have improved physical properties (e.g. tabletability and solubility), and may improve patient compliance due to the relative ease of taking a single combined dose of the two APIs.

Suitably the lamotrigine-valproic acid multicomponent crystal comprises an ionic form and a neutral form of the lamotrigine and an ionic form and a neutral form of the valproic acid.

In some embodiments of the multicomponent crystal of this first aspect, the first API is lamotrigine and/or a lamotrigine salt and the second API is benzoic acid and/or a benzoic salt. In such embodiments, the multicomponent crystal of lamotrigine and benzoic acid may provide a monotherapy for the treatment of epilepsy, suitably having improved physical properties (e.g. tabletability and solubility), as discussed above, compared to known dosage forms of lamotrigine. Suitably the lamotrigine-benzoic acid multicomponent crystal comprises an ionic form of the lamotrigine and an ionic form and a neutral form of the benzoic acid.

According to a second aspect of the present invention, there is provided a multicomponent crystal according to the first aspect, for use as a medicament.

The multicomponent crystal for use as a medicament of this second aspect may have any of the suitable features and advantages described in relation to the first aspect. The multicomponent crystal may be used in the treatment of any disorder, which the first and second APIs are suitable for treating.

According to a third aspect of the present invention, there is provided a multicomponent crystal according to the first aspect, for use in the treatment of epilepsy. The multicomponent crystal of this third aspect may have any of the suitable features and advantages described in relation to the first aspect. Suitably the first API is lamotrigine and/or a lamotrigine salt and the second API is valproic acid and/or a valproate salt and the molar ratio of the lamotrigine and/or a lamotrigine salt to the valproic acid and/or a valproate salt in the multicomponent crystal is 1:2.

According to a fourth aspect of the present invention, there is provided a pharmaceutical composition comprising a therapeutically effective amount of a multicomponent crystal according to the first, second or third aspects, and a pharmaceutically acceptable excipient.

According to a fifth aspect of the present invention, there is provided a method of preparing a multicomponent crystal comprising at least two active pharmaceutical ingredients, the method comprising the steps of:

a) providing a first active pharmaceutical ingredient comprising a nitrogen-containing heterocycle substituted with an amino group;

b) providing a second active pharmaceutical ingredient comprising a carboxylic acid; and

c) combining the first active pharmaceutical ingredient and the second active pharmaceutical ingredient;

d) crystallising the combination of the first active pharmaceutical ingredient and the second active pharmaceutical ingredient obtained from step c) to provide the multicomponent crystal.

The multicomponent crystal formed by the method of this fifth aspect may have any of the suitable features or advantages described in relation to the first aspect.

Suitably the multicomponent crystal comprises an interaction between the nitrogen-containing heterocycle substituted with an amino group of the first active pharmaceutical ingredient and the carboxylic acid of the second active pharmaceutical ingredient.

Suitably step a) involves providing a powder of the first active pharmaceutical ingredient.

Suitably step b) involves providing a powder of the second active pharmaceutical ingredient.

Suitably step c) involves combining powders of the first active pharmaceutical ingredient and the second active pharmaceutical ingredient.

Step d) may involve forming a slurry of the first active pharmaceutical ingredient and the second active pharmaceutical ingredient and then drying to form a microcrystalline powder.

Suitably step d) involves, suitably after the formation of the slurry and drying, of dissolving in a solvent the first active pharmaceutical ingredient and the second active pharmaceutical ingredient and evaporating the solvent, to form the multicomponent crystal.

The present invention may also be applicable where a crystalline form of a non-pharmaceutically active composition is required. Therefore according to a further aspect of the present invention, there may be provided a multicomponent crystal of a first compound and a second compound;

wherein the first compound comprises a nitrogen-containing heterocycle substituted with an amino group;

wherein the second compound comprises a carboxylic acid; and

wherein the nitrogen-containing heterocycle substituted with an amino group of the first compound interacts with the carboxylic acid of the second compound, in the multicomponent crystal.

The multicomponent crystal of this further aspect may have any of the suitable features and advantages described above in relation to the first aspect.

Suitably the multicomponent crystal of this further aspect comprises a neutral form of at least one of the first or second compounds, or any third compound present. Suitably the neutral form of said compound stabilises the crystal structure, suitably by stabilising the the R₁² (4) and R₂²(8) motif discussed above, suitably by H-bonding.

In some embodiments, the multicomponent crystal comprises an ionic form and a neutral form of the first compound and an ionic form of the second compound.

In some embodiments, the multicomponent crystal comprises an ionic form and a neutral form of the second compound and an ionic form of the first compound.

In some embodiments, the multicomponent crystal comprises an ionic form and a neutral form of the first compound and an ionic form and a neutral form of the second compound; wherein the first compound and the second compound are organic compounds.

Suitably the interaction of the nitrogen-containing heterocycle substituted with an amino group of the first compound and the carboxylic acid group of the second compound comprises an R₁² (4) synthon.

Suitably the interaction of the nitrogen-containing heterocycle substituted with an amino group of the first compound and the carboxylic acid group of the second compound comprises an R₂² (8) synthon.

### EXAMPLES

Materials and Methods

In the following description, the comparative example lamotrigine (single API) is termed “LAM” and the multicomponent crystal of lamotrigine and valproic acid is termed “LAMVAL”.

Lamotrigine and valproic acid (also termed “VAL” herein) were obtained from Baoji Guokang Bio-Technology Co. Ltd. and used without further purification. All other solvents and reagents were purchased from Sigma-Aldrich and used as received.

### Example 1—Lamotrigine:Valproic Acid

Synthesis of lamotrigine:valproic acid (1:2) ionic cocrystal (LAMVAL): 256.09 mg of lamotrigine (1 mmol) and 1.82 ml of valproic acid (2 mmol) were placed in a mortar and pestle and manually ground for 5 minutes until a dry, fine white powder was produced. Synthesis was also attempted by ball milling the same reagents in Retsch MM400 shaker mill in a 15 mL steel vessel with one 15 mm steel ball at 25 hz for 15 min.

Slurry experiments were conducted by stirring 256.09 mg of lamotrigine (1 mmol) and 1.82 ml of valproic acid (2 mmol) in deionised water (10 ml) for 48 h. The product was recovered by filtration and dried in air.

Recrystallization was attempted by slow evaporating a solution of about 5 mg of the microcrystalline powder in 20 ml of ethanol in a vial and by slow cooling of a hot solution containing 5 mg of the microcrystalline powder in 20 ml of 1:1 isopropanol/methanol (vide infra).

Powder X-ray diffraction (PXRD): X-ray powder diffraction (XRPD) patterns were collected in Bragg-Brentano geometry on a PANalytical Empyrean diffractometer equipped with a sealed tube (Cu Kα₁₂, λ=1.5418 Å) an 1D X'Celerator detector between 4 and 40° 2θ.

Variable temperature PXRD data were collected in Bragg-Brentano geometry on a X'Pert MPD Pro equipped with a Anton-Paar TK450 stage, a sealed tube (Cu Kα₁₂, λ=1.5418 Å) and a 1 D X'Celerator detector in the 4-30° (2θ) range.

Single-crystal X-ray Diffraction: Single crystals were manually selected and mounted with Paratone® oil on a polymeric fibre. Data was collected at room temperature (298 K) as well as at 100 K on a Bruker Quest D8 diffractometer equipped with Mo sealed tube Tube (Mo-Kα radiation λ=0.71073 Å), a Photon II CPAD detector and Oxford Cryosystem Cryostreem 800. Data was integrated with the APEX program suite and empirically corrected for absorption correction. Structure solution was found through direct methods in SHELX through X-Seed. All heavy atoms were found on the electron density map and refined anisotropically against all F²_{obb}. H atoms were constrained through the riding model in their position as determined by an analysis of the distances between heavy atoms.

Results and Discussion

Crystal Synthesis and Structure Analysis

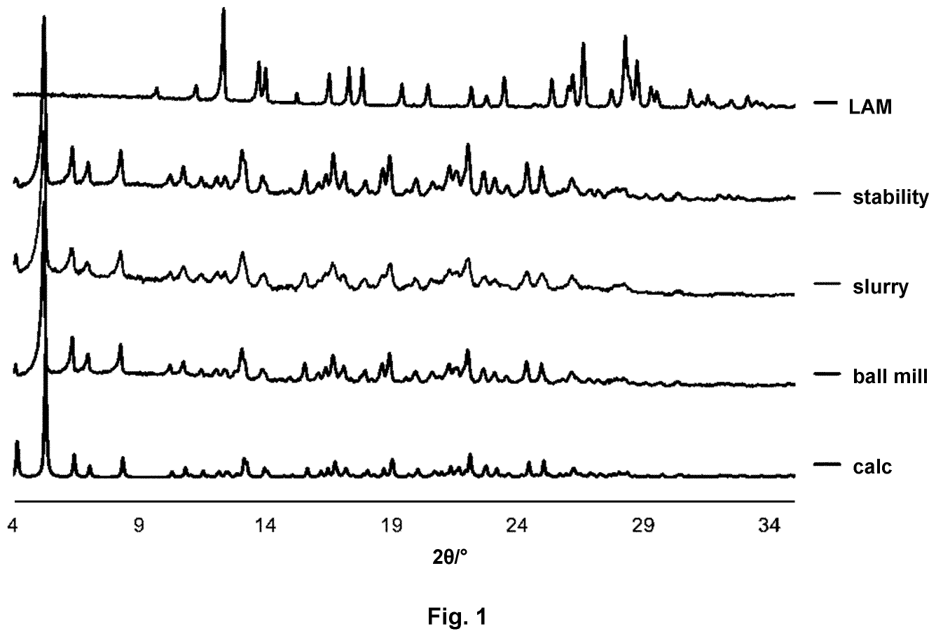

The neat grinding described above of a 1:2 ratio of LAM and VAL in a ball mill affords a dry microcrystalline powder. PXRD reveals that the product is stable as a slurry in water. Recrystallization by slow evaporation from ethanol resulted in crystals of a lamotrigine ethanolate (CSD ref. code GEVYOY). Recrystallization by slow cooling from isopropanol/methanol, affords quality single crystals of the title multicomponent crystal: LAMVAL. The monoclinic P2,1n unit cell comprises two LAM and four VAL independent residues. PXRD confirms the identity between the bulk powder and the single crystal, as shown in FIG. 1 wherein the traces in order from the top show the PXRD data for lamotrigine; LAMVAL after stability test; LAMVAL after slurring in water; LAMVAL by ball mill; and calculated from LAMVAL single crystal data.

Since traditional X-ray diffraction cannot determine the hydrogen position in a reliable manner, the values of C—O distances were used to confirm that one LAM and one VAL species are ionised forming an ionic or salt multicomponent crystal. Crystals forms in which both components have mixed ionization state are rarely seen. LAMVAL may be considered to belong to a novel type of multicomponent crystal that could be represented as (aA)A*B⁻(bB).

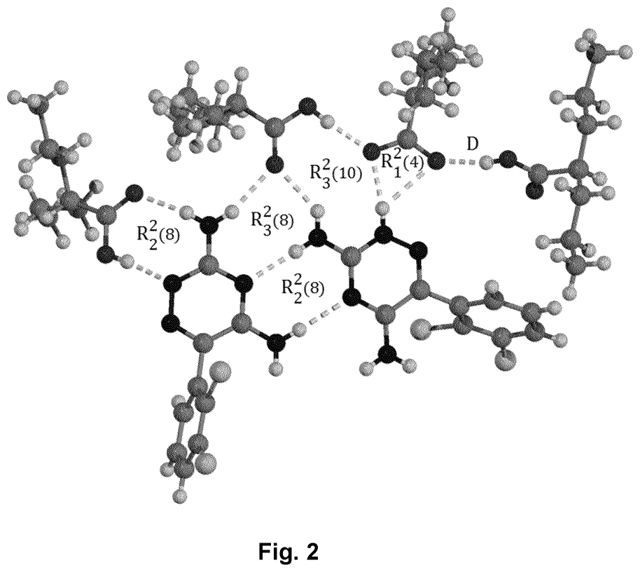

FIG. 2 shows a graph set analysis of LAMVAL highlighting key intermolecular interactions. The supramolecular structure is rather complex and multiple motifs can be recognised. One VAL molecule forms a typical R₂²(8) heterosynthon with the neutral LAM through the N2 donor. Another R₂²(8) motif forms between the aminopyridine moiety of the neutral LAM and the aminopyridine moiety of the cationic LAM involving both the N4 donors, as shown in FIG. 2. Notably the triazine rings in the synthon lay on different planes. The carbonyl oxygen of a VAL stabilises the interaction by bridging the adjacent amine hydrogens: R₃²(8). The same VAL also donates into a charged assisted H-bond with the valproate generating a R₃²(10) motif. In the cation, the proton sits on the most basic nitrogen (N2) and a double, charge-assisted H-bond forms with the carboxylate anion: R₁²(4) motif. Finally, the last VAL donates into the valproate (D), and bridges with an adjacent complex to guarantee 1D H-bonded structure along the b crystallographic direction (FIG. 2).

From a supramolecular point of view, the R₁² (4) motif is unusual. A CSD analysis revealed only another example of such motif (CSD ref. code VECVAD) out of 349 total entries containing aminopyridinium-carboxylate interactions (see methods). In all the other cases, the typical R₂²(8) motif is present, which involves both the pyridinium and the amino functions.

A CSD search of the multicomponent crystals that include LAM with carboxylic groups revealed that in 43 out of 48 cases LAM is protonated in the N2 position. In the remaining five cases (CSD ref. codes HUQIVA QIQHIJ QIQHOP WOKXUR GAVLEV), C—O bond distance analysis suggest that a partial charge transfer is present: the proton is reported either disordered over the nitrogen and oxygen positions or sitting half way. Such behaviour is consistent with the aforementioned idea of a continuum between neutral and ionised forms.

A visual inspection reveals that in all the structures with partial charge transfer, the R₂²(8) is always isolated. On the contrary, in the structures reported as salts, the carboxylate is always involved in at least another H-bond: with carboxylic, amine or hydroxyl donors. Indeed, a CSD search revealed other 62 cases of the same charge-assisted synthon involving either three or four functional groups in structures containing α-aminopyridines, α-aminopyrazine, or α-aminotriazine with carboxylic acids.

These observations suggest that the ionic character of the synthons could be related to the presence of ancillary H-bond donors. Such behaviour is exemplified by two forms of LAM and acetic acid (AA). LIBXUR is an ionic multicomponent crystal of LAM and AA in a 1:3 ratio that shows the same synthon as LAMVAL. In the new LAMAA 1:1 salt, two independent ionic R₂²(8) synthon are complemented by an H-bonded amine group each. In these cases, the difference in C—O distances 0.003(2), 0.001(2) and 0.045(2), suggest that the degree of ionization decreases with the H-bond donor distance: the 2.553(2), 2.752(2) and 2.912(2) respectively.

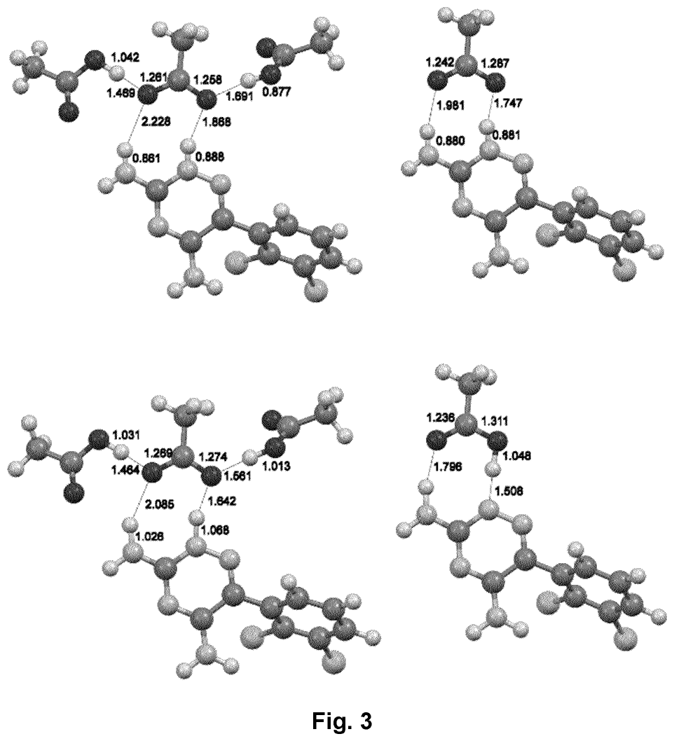

Such hypothesis was further confirmed computationally. The refinement of hydrogen positions in the four-molecular synthon of LIBXUR confirms that the ionic multicomponent crystal is the most stable form. On the contrary when the same refinement is performed on the isolated two-molecular R₂²(8) synthon, the multicomponent crystal form is the favoured (FIG. 3). FIG. 3 shows the following—Top: molecular and supramolecular distances as measured by single crystal XRD data for LIBXUR (left) and LAMAA (right). Bottom: molecular and supramolecular distances as measured from the DFT refined model LIBXUR (left) and LAMAA (right).

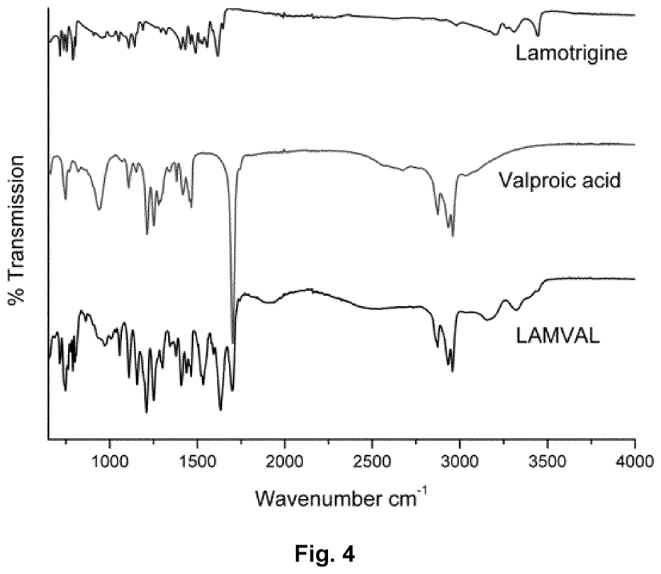

Fourier-transform infrared spectroscopy (FTIR) was employed to investigate the mechanism behind the solid state changes in the powder material. FIG. 4 illustrates major peaks relating to presence of starting materials and demonstrated changes within the fingerprint region, suggestive of the formation of a new solid form. Of note, there is a shift of 1609 peak in LAM to 1638 in LAMVAL related to amine protons, a more pronounced peak at 1540 in LAMVAL, at 1444 slight peak change in LAMVAL, a new peak at 1058 in LAMVAL, 936 band missing in LAMVAL replaced by new band at 905-1005 and the increase of the 666 peak LAMVAL. All of these changes could be related to the formation of intermolecular H-bonds.

Survey of Cambridge Structural Database (CSD): The Cambridge Structural Database was searched through ConQuest (v. 1.23, 2018) and the retrieved entries were subsequently analysed with Mercury (v. 3.10.3, 2018). In all cases, the cut off distance for the interatomic interactions between H and O was set equal to the sum of vdW radii+0.3 Å. This precaution was justified by the possible errors associated with the H position.

Quantumechanical calculation: Computational studies were performed with GAUSSIAN 09. Model structures were created starting from the crystallographic data replacing the propyl groups on each valproic acid with H atoms. The coordinates of all the H and O atoms plus those of selected C and N were refined by M06-2X/6-31+G(d,p) level of theory.

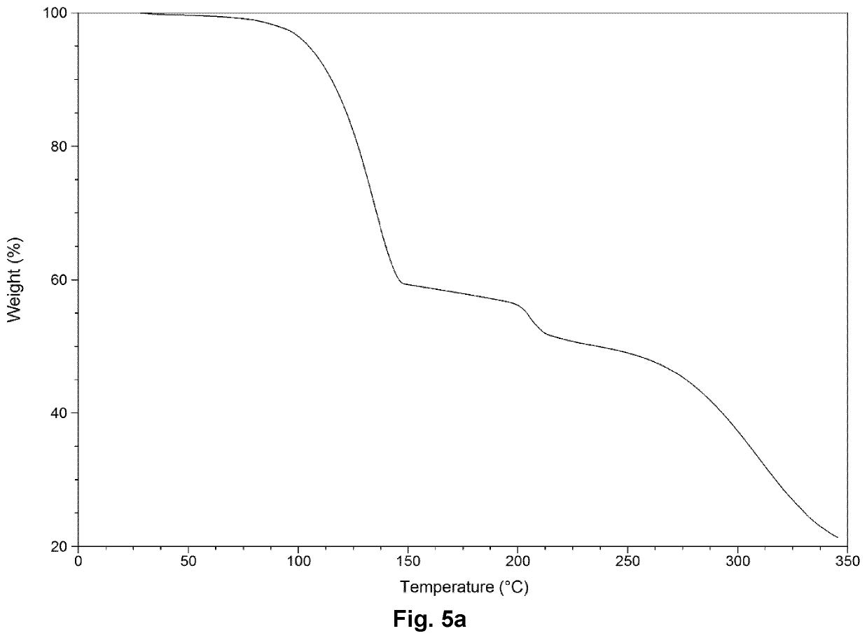

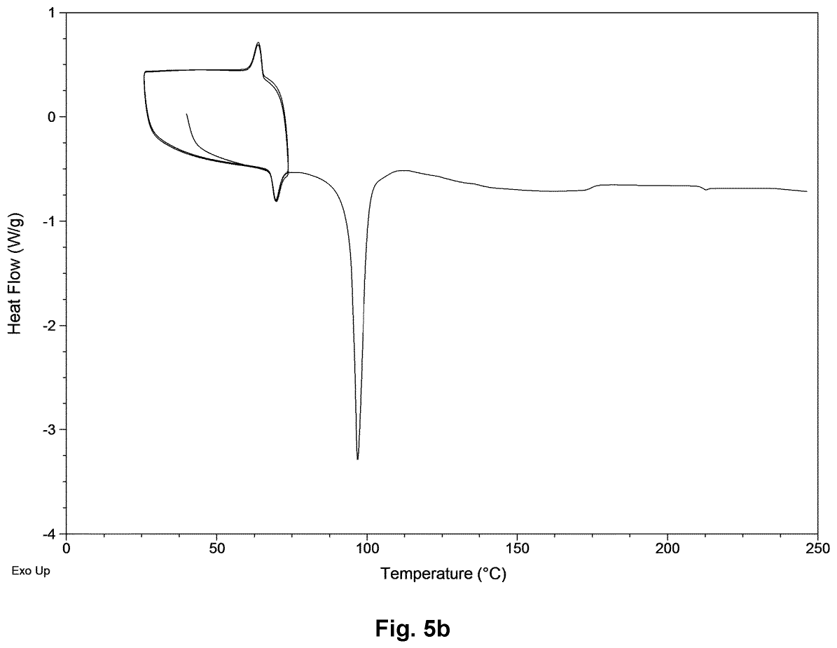

Thermal Analysis: Thermogravimetric analysis (TGA) was performed using a TA Instruments TGA-Q50 on at a constant rate of 10° C./min from 25° C. to 350° C. under a flux of nitrogen of 50 ml/min. Differential Scanning Calorimetry (DSC) was carried out using sealed aluminium pans on a TA instruments DSC-Q2000 differential scanning calorimeter. Temperature calibrations were made using indium as the standard. An empty pan, sealed in the same way as the sample, was used as a reference. All the thermograms were run at a heating/cooling rate of 10° C./min under a nitrogen purge at a rate of 50 ml/min. FIG. 5 shows the TGA (top) and DSC (bottom) traces for LAMVAL.

Scanning Electron Microscopy (SEM): LAM and LAMVAL were separately placed onto carbon tape and coated with a thin layer of gold followed by analysis on a Joel CarryScope JCM-5700 scanning electron microscope. Micrographs were recorded at various magnifications using a beam voltage of 2.0 kV.

Intrinsic solubility study: Compacts of LAM and LAMVAL containing equivalent amounts of LAM were made by compacting 100 mg LAM and 220 mg of LAMVAL in an 8 mm punch and die set for 3 minutes using a hydraulic press with a compaction force of 5 tonnes. These compacts were each coated with paraffin wax, leaving one surface exposed and secured to the bottom of the dissolution apparatus with excess paraffin wax. Intrinsic solubility was determined using a 900 ml well filled with 900 ml of degassed, deionised water (or 0.1 M HCl solution) on a Pharma Test USP type II system. The solution had been previously equilibrated at 37° C. and the paddle speed was set to 100 rpm after adding the samples. Aliquots of media were withdrawn at 5, 10, 15, 20 30 and 60 minutes (or 0.25, 0.5, 1, 3, 6, 12 and 24 hours), filtered through 0.45 μm PTFE filters and tested via a UV spectrometer in triplicate.

Spectrometry: Infrared analysis was performed on a Perkin-Elmer Spectrum 100 FT-IR spectrometer equipped with a solid-state ATR stage. UV-Vis absorbance measurements were carried on a Cary 60 UV-Vis spectrometer using 1 ml quartz cuvettes. Calibration curves were obtained by linear regression from a set of absorbance measurements from solutions on lamotrigine of known concentration preformed in triplicate.

Tensile stress test: Tensile strength analysis for lamotrigine and LAMVAL was conducted on tablets produced from 100 mg of milled powder (25 hz, 15 m) across a range of compaction forces using a 6 mm, flat-faced punch and die, using a Gamblen R-series tablet press. Hardness testing was performed immediately after tableting using a Pharma Test hardness tester. Each compaction force was tested in triplicate. Tensile strength was calculated according to the Equation 1 where F is the load required to fracture the tablet, D the table diameter and H the tablet height:

Tensile strength=2F/πDH Equation 1:

Stability testing: Accelerated stability testing was conducted by taking 100 mg of powdered sample and placing it into a humidity chamber under 75% relative humidity at 40° C. for 14 days. The samples were analysed before and after testing by PXRD, FT-IR, DSC and TGA.

Physicochemical Characterization

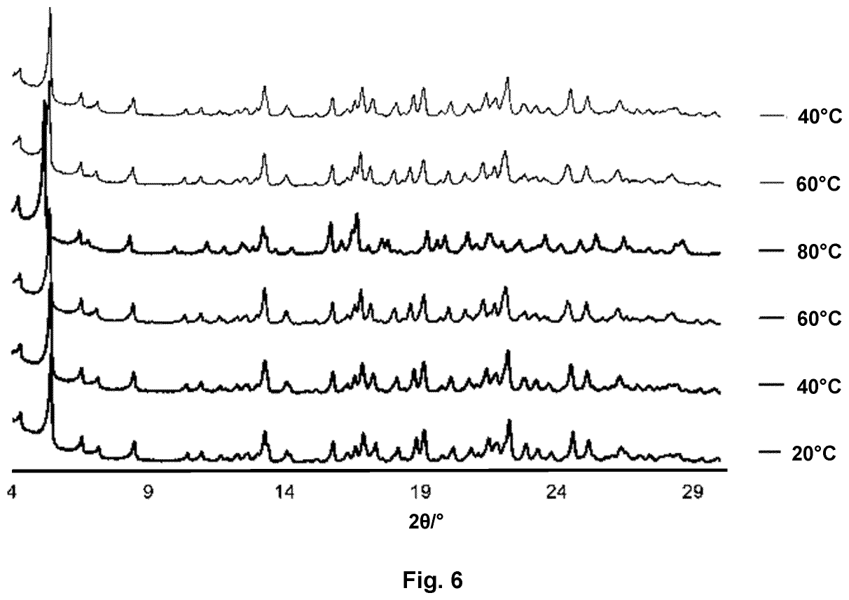

Thermogravimetric analysis shows that LAMVAL is thermally stable up to around 100° C. Differential scanning calorimetry and variable temperature PXRD reveals that the material undergoes an enantiotropic transition above 75° C. (see FIG. 6). FIG. 6 shows the variable temperature PXRD for LAMVAL.

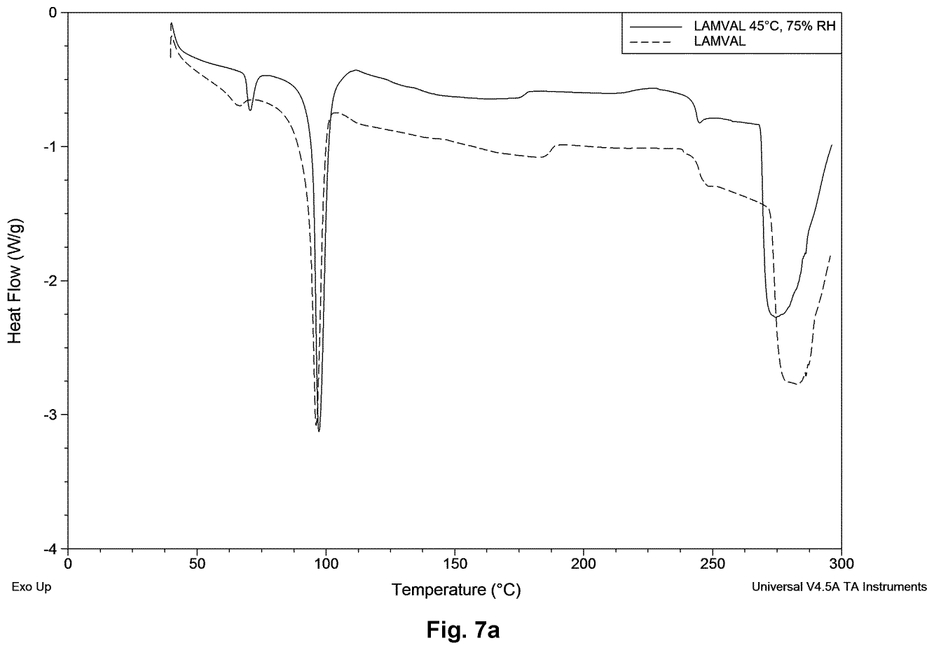

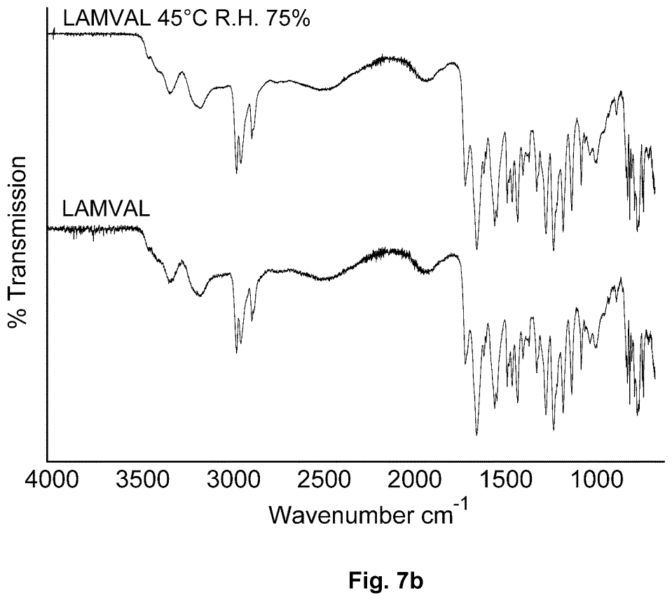

LAMVAL powder remains crystalline at 45° C. at 75% relative humidity for two weeks (see FIG. 1, second line from the top), and FT-IR and DSC (FIG. 7) show no sign of product degradation. FIG. 7 shows DSC (top) and FTIR (bottom) before and after accelerated stability testing.

Pharmaceutical Characterization

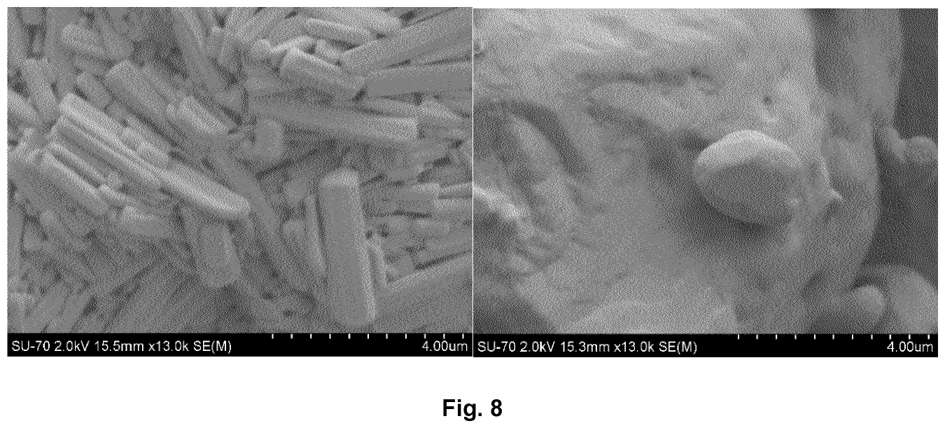

Scanning electron microscopy shows that a polycrystalline powder of LAMVAL is formed by clusters of smaller prisms whereas LAM forms large, smooth crystals (see FIG. 8). FIG. 8 shows SEM images of LAMVAL (left) and Lamotrigine (Right). It is well-known that crystal morphology can affect the mechanical properties of a material. The tensile strength was measured for the two materials.

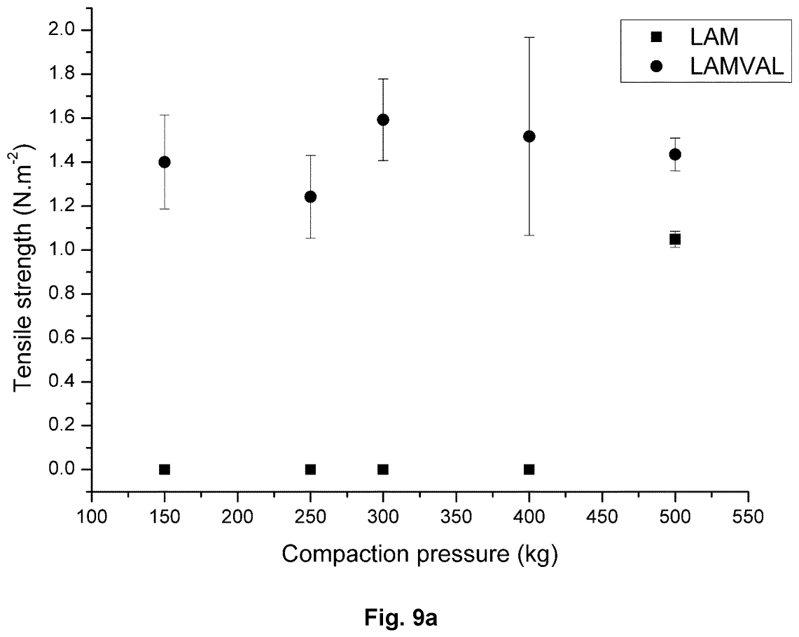

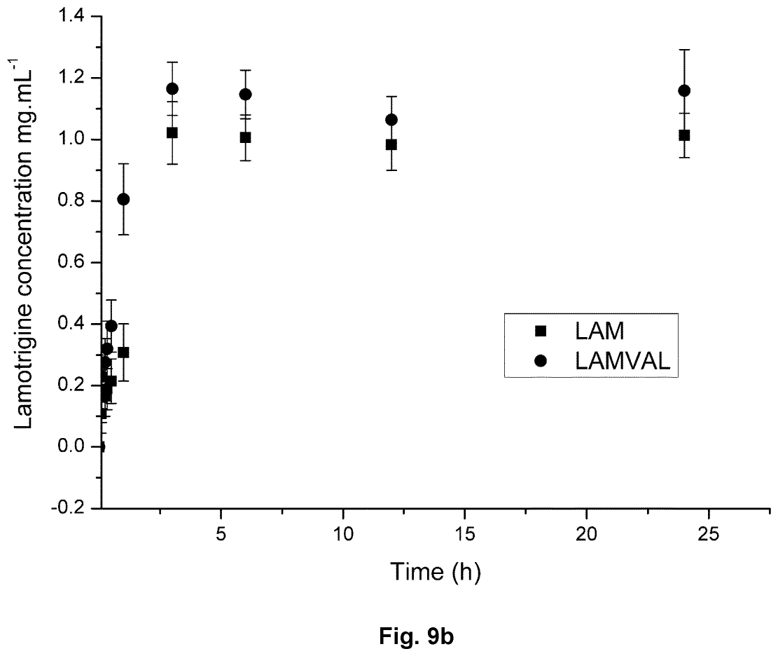

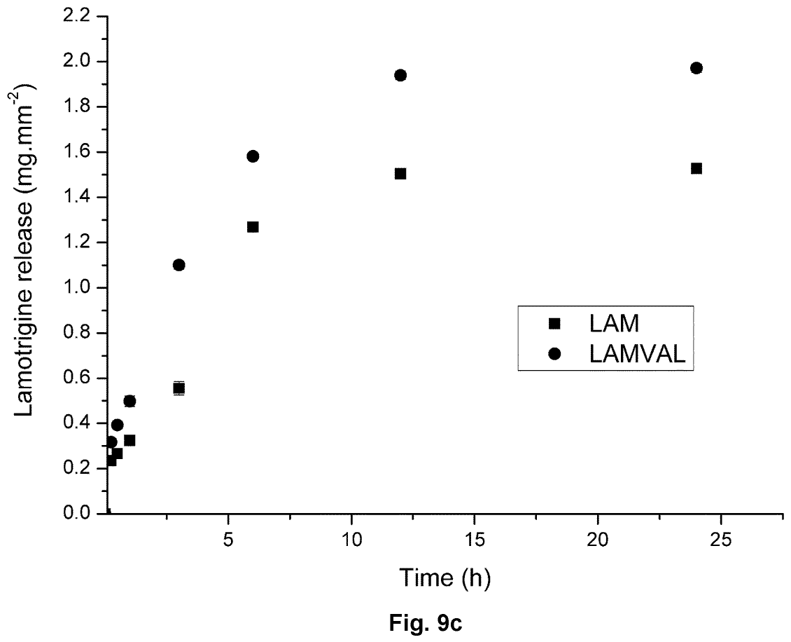

Tablets of LAM and LAMVAL were prepared by compressing the polycrystalline powders at different pressure. Tablets of LAM obtained with a compaction weight lower than 400 kg were too brittle to enable hardness testing. Tablets of LAMVAL obtained in the same conditions demonstrate superior tableting properties with significantly greater tensile strength across a range of compaction forces (FIG. 9). FIG. 9 shows a comparison of physicochemical properties LAMVAL (circular data points) and pure Lamotrigine (square data points): top—tensile strength; middle—dissolution rate in PBS buffer (pH=7.4); bottom—dissolution rate in 0.1 M HCl solution (pH=1.2). In the bottom graph, the error bars are smaller than the data points.

Direct compression is the preferred method of tablet processing and enables this formulation to fit into continuous manufacturing processes, an area of particular interest for industry. Materials that show poor tabletting properties can be formulated with excipients that act as binders. Although effective, such procedures increase the size of the dosage form, a particular concern for multidrug formulations such as this. The improved mechanical properties of LAMVAL suggest that tablets could be manufactured with minimal use of excipients, which would increase the tablet size.

Measurements of intrinsic dissolution show that the ionic multicomponent crystal LAMVAL dissolves significantly (×2) faster than pure LAM (see FIG. 9). In PBS buffer, at pH 7.4, the ionic multicomponent crystal affords a concentration of lamotrigine 20% higher than the pure base. In physiological acidic conditions (pH 1.2 M) LAMVAL affords a 33% increase in lamotrigine concentration.

Lamotrigine is a BCS class II drug, whose bioavailability is limited by poor solubility. Attempts at crystal engineering have been reported that aimed to find forms that are more soluble. In those cases, the formation of neutral adducts resulted in a material less soluble than LAM. On the contrary, LAMVAL produced a higher dissolution rate in in vitro physiological conditions, which could translate into increased bioavailability, without the need for excipients and therefore avoiding an increase in the size of the dosage form.

### CONCLUSIONS

A stable ionic multicomponent crystal of LAM and VAL has been obtained either mechano-chemically or from solution in a reliable manner. The physicochemical properties such as dissolution rate and tabletability are significantly higher than those of LAM alone. Most importantly, the 1:2 stoichiometry appears to improve the pharmacokinetics of the APIs making LAMVAL an ideal candidate for a marketable multidrug dosage form.

The LAMVAL structure is sustained by a complex set of supramolecular motifs that include a four-component ionic synthon between the pyridinium a carboxylate and two ancillary carboxylic acids.

### Example 2—Lamotrigine:Benzoic Acid

Synthesis of lamotriginium:benzoate:benzoic acid ionic cocrystal: 25.6 mg of lamotrigine (0.1 mmol) and 24.4 mg of benzoic acid (0.2 mmol) in were dissolved in a 20 ml solution of 1:1 isopropanol/methanol by heating and recrystallization was attempted by slow cooling.

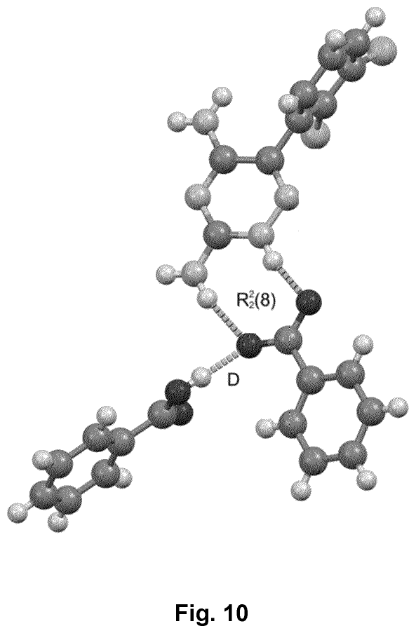

Single-crystal X-ray Diffraction: X-ray diffraction data was collected and analysed as described above for Example 1, apart from data here being collected at room temperature and 150 K on the Bruker Quest D8 diffractometer, to provide the crystal structure shown in FIG. 10 and discussed below.

Results: FIG. 10 displays graph-set analysis of the ionic cocrystal of Example 2. One benzoic acid cation forms a R₂²(8) heterosynthon with the aminopyridinum group in lamotrigine resulting in proton transfer from benzoic acid to lamotrigine. The proton is located on the most basic nitrogen (N2) as seen previously. Another neutral benzoic acid donates into the benzoate (D) to stabilise this charged interaction.

### Example 3—Lamotrigine:Benzoate:Isopropanol

Synthesis of lamotriginium:benzoate:isopropanol: 25.6 mg of lamotrigine (0.1 mmol) and 12.2 mg of benzoic acid (0.1 mmol) and excess acetic acid (1 ml) were dissolved in a 20 ml solution of 1:1 isopropanol/methanol by heating and recrystallization was attempted by slow cooling.

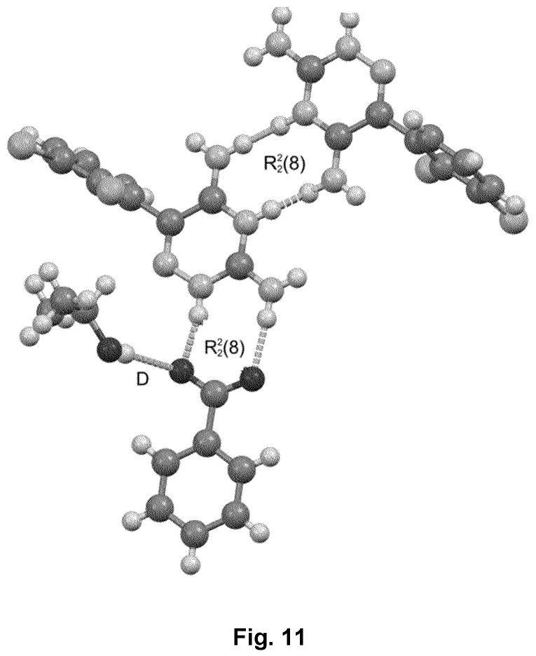

Single-crystal X-ray Diffraction: The crystal structure shown in FIG. 11 and discussed below was obtained for this Example 3 as described above for Example 2.

Results: FIG. 11 displays graph-set analysis of the ionic cocrystal of Example 3. As in Example 2, one benzoic acid cation forms a R₂²(8) heterosynthon with the aminopyridinum group in lamotrigine resulting in proton transfer from benzoic acid to lamotrigine. The proton is located on the most basic nitrogen (N2) as described previously. However, this time the charge transfer is stabilised by isopropanol.

### Example 4—Melaminium:Acetate:Acetic Acid Hydrate Cocrystal

Synthesis of Melaminium:acetate:acetic acid hydrate cocrystal: 12.6 mg of melamine (0.1 mmol) and excess acetic acid (1 ml) were dissolved in a 20 ml solution of 1:1 isopropanol/methanol by heating and recrystallization was attempted by slow cooling.

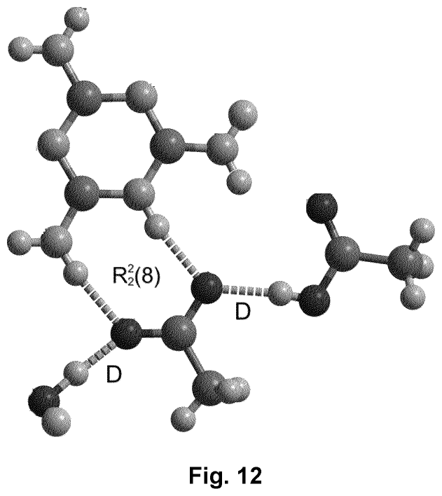

Single-crystalX-ray Diffraction: The crystal structure shown in FIG. 12 and discussed below was obtained for this Example 4 as described above for Example 2.

Results: FIG. 12 displays graph-set analysis of the ionic cocrystal of Example 4. Here, one acetic acid cation forms a R₂²(8) heterosynthon with the aminopyridinum group in melamine resulting in proton transfer from acetic acid to melamine. The proton is located on the most basic nitrogen. Another neutral acetic acid in addition to a water molecule donates into the acetate (D) to stabilise this charged interaction.

The present invention demonstrates that the use of high-order synthons can provide ionic multicomponent crystals between heterocycle-amine and carboxylic acids groups in different APIs to enable the preparation of multidrug dosage forms with improved drug properties.

In summary, the present invention provides a multicomponent crystal (or co-crystal) comprising a first active pharmaceutical ingredient and a second active pharmaceutical ingredient. The multicomponent crystal is formed/sustained by non-covalent interactions between the nitrogen-containing heterocycle alpha-substituted with an amino group of the first active pharmaceutical ingredient and a carboxylic acid group of the second active pharmaceutical ingredient, suitably as well as other further non-covalent interactions with other H-bond forming groups. The multicomponent crystal may provide an improved multidrug dosage form comprising lamotrigine and valproic acid as the first and second active pharmaceutical ingredients, respectively. A pharmaceutical composition comprising a therapeutically effective amount of the multicomponent crystal and a pharmaceutically acceptable excipient is also provided.

As used herein, the term “alkyl” means both straight and branched chain saturated hydrocarbon groups. Examples of alkyl groups include methyl, ethyl, n-propyl, iso-propyl, n-butyl, t-butyl, i-butyl, and sec-butyl groups.

As used herein, the term “cycloalkyl” means a cyclic saturated hydrocarbon group. Examples of cycloalkyl groups include cyclopropyl, cyclobutyl, cyclopentyl and cyclohexyl.

As used herein, the term “aryl” means a carbocyclic aromatic system.

As used herein, the term “heteroaryl” means a cyclic aromatic system comprising at least one carbon atom and at least one heteroatom, for example at least one nitrogen atom.

As used herein, the term “halogen” or “halo” means fluorine, chlorine, bromine or iodine. Fluorine, chlorine and bromine are particularly preferred.

“Pharmaceutically acceptable salt” means a salt such as those described in standard texts on salt formation, see for example: P. Stahl, et al., Handbook of Pharmaceutical Salts: Properties, Selection and Use (VCHA/WNey-VCH, 2002), or S. M. Berge, et al., “Pharmaceutical Salts” (1977) Journal of Pharmaceutical Sciences, 66, 1-19. Suitable salts according to the invention include those formed with organic or inorganic acids or bases. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as C₁-C₄alkyl- or aryl-sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Other acids, which may or may not in themselves be pharmaceutically acceptable, may be useful as intermediates in obtaining the compounds of the invention and their pharmaceutical acceptable acid addition salts.

Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. Corresponding internal salts may furthermore be formed.

“Pharmaceutically acceptable solvate” means a molecular complex comprising the compound of the invention and one or more pharmaceutically acceptable solvent molecules, for example, water or ethanol. Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates, such as hydrates, exist when the drug substance incorporates solvent such as water, in the crystal lattice in either stoichiometric or non-stoichiometric amounts. Drug substances are routinely screened for the existence of hydrates since these may be encountered at any stage of the drug manufacturing process or upon storage of the drug substance or dosage form. Solvates are described in S. Byrn et al., Pharmaceutical Research, 1995. 12(7): p. 954-954, and Water-Insoluble Drug Formulation, 2<nd>ed. R. Liu, CRC Press, page 553, which are incorporated herein by reference.

“Therapy”, “treatment” and “treating” include both preventative and curative treatment of a condition, disease or disorder. It also includes slowing, interrupting, controlling or stopping the progression of a condition, disease or disorder. It also includes preventing, curing, slowing, interrupting, controlling or stopping the symptoms of a condition, disease or disorder.

Although a few preferred embodiments have been shown and described, it will be appreciated by those skilled in the art that various changes and modifications might be made without departing from the scope of the invention, as defined in the appended claims.

Throughout this specification, the term “comprising” or “comprises” means including the component(s) specified but not to the exclusion of the presence of other components. The term “consisting essentially of” or “consists essentially of” means including the components specified but excluding other components except for materials present as impurities, unavoidable materials present as a result of processes used to provide the components, and components added for a purpose other than achieving the technical effect of the invention. Typically, when referring to compositions, a composition consisting essentially of a set of components will comprise less than 5% by weight, typically less than 3% by weight, more typically less than 1% by weight of non-specified components.

The term “consisting of” or “consists of” means including the components specified but excluding addition of other components.

Whenever appropriate, depending upon the context, the use of the term “comprises” or “comprising” may also be taken to encompass or include the meaning “consists essentially of” or “consisting essentially of”, and may also be taken to include the meaning “consists of” or “consisting of”.

The optional features set out herein may be used either individually or in combination with each other where appropriate and particularly in the combinations as set out in the accompanying claims. The optional features for each aspect or exemplary embodiment of the invention as set out herein are also to be read as applicable to any other aspect or exemplary embodiments of the invention, where appropriate. In other words, the skilled person reading this specification should consider the optional features for each exemplary embodiment of the invention as interchangeable and combinable between different exemplary embodiments.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, and drawings), and/or all of the steps of any method or process so disclosed, may be combined in any combination, except combinations where at least some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, and drawings), or to any novel one, or any novel combination, of the steps of any method or process so disclosed.

1. A multicomponent crystal of a first active pharmaceutical ingredient and a second active pharmaceutical ingredient;
wherein the first active pharmaceutical ingredient comprises a nitrogen-containing heterocycle substituted with an amino group;
wherein the second active pharmaceutical ingredient comprises a carboxylic acid group; and
wherein the nitrogen-containing heterocycle substituted with an amino group of the first active pharmaceutical ingredient interacts with the carboxylic acid of the second active pharmaceutical ingredient, in the multicomponent crystal.

2. The multicomponent crystal according to claim 1, wherein the first active pharmaceutical ingredient and/or the second active pharmaceutical ingredient form non-covalent interactions with other H-bond forming groups of the components of the multicomponent crystal.

3. The multicomponent crystal according to claim 1, wherein the nitrogen-containing heterocycle substituted with an amino group of the first active pharmaceutical ingredient has the structure (I): 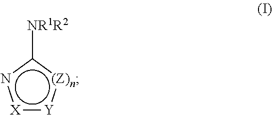
wherein R¹ and R² are each independently selected from H, a C₁-C₈ alkyl, a C₁-C₈ alkenyl, an aryl group, an alkylaryl group, a heteroaryl group or an alkylheteroaryl group, optionally substituted with one or more of C₁-C₄ alkoxy, hydroxy, amino, carboxylic acid, ester, amide, halogen, CF₃, CHF₂ or CH₂F groups;
wherein n=0, 1, 2 or 3;
wherein X, Y and each Z are independently selected from N or C atoms;
wherein said N atoms are optionally substituted with a C₁-C₈ alkyl, a C₁-C₈ alkenyl, an aryl group, an alkylaryl group, a heteroaryl group or an alkylheteroaryl group, which are optionally substituted with one or more of C₁-C₄ alkoxy, hydroxy, amino, carboxylic acid, ester, amide, halogen, CF₃, CHF₂ or CH₂F groups; and
wherein said C atoms are optionally substituted with C₁-C₈ alkyl, a C₁-C₈ alkenyl, an aryl group, an alkylaryl group, a heteroaryl group or an alkylheteroaryl group, optionally substituted with one or more of C₁-C₄ alkoxy, hydroxy, amino, carboxylic acid, ester, amide, halogen, CF₃, CHF₂ or CH₂F groups, or wherein said C atoms are optionally substituted with NR⁵R⁶, wherein R⁵ and R⁶ are each independently selected from H, a C₁-C₄ alkyl, a C₁-C₄ alkenyl, an aryl group, an alkylaryl group, a heteroaryl group or an alkylheteroaryl group, optionally substituted with one or more of C₁-C₄ alkoxy, hydroxy, amino, carboxylic acid, ester, amide, halogen, CF₃, CHF₂ or CH₂F groups.

4. The multicomponent crystal according to claim 1, wherein the nitrogen-containing heterocycle substituted with an amino group of the first active pharmaceutical ingredient has the structure (II): 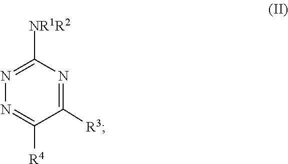
wherein R¹ and R² are each independently selected from H, a C₁-C₈ alkyl, a C₁-C₈ alkenyl, an aryl group, an alkylaryl group, a heteroaryl group or an alkylheteroaryl group, optionally substituted with one or more of C₁-C₄ alkoxy, hydroxy, amino, halogen, CF₃, CHF₂ or CH₂F groups;
wherein R³ and R⁴ are each independently selected from H, NR⁵R⁶, C₁-C₈ alkyl, a C₁-C₈ alkenyl, an aryl group, an alkylaryl group, a heteroaryl group or an alkylheteroaryl group, optionally substituted with one or more of C₁-C₄ alkoxy, hydroxy, amino, carboxylic acid, ester, amide, halogen, CF₃, CHF₂ or CH₂F groups; wherein R⁵ and R⁶ are each independently selected from H, a C₁-C₄ alkyl, a C₁-C₄ alkenyl, an aryl group, an alkylaryl group, a heteroaryl group or an alkylheteroaryl group, optionally substituted with one or more of C₁-C₄ alkoxy, hydroxy, amino, carboxylic acid, ester, amide, halogen, CF₃, CHF₂ or CH₂F groups.

5. The multicomponent crystal according to claim 1, wherein the second active pharmaceutical ingredient has the 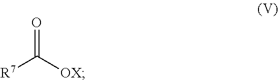
structure (V):
wherein X is H or a negative charge; and
wherein R⁷ is selected from C₁-C₁₀ alkyl, a C₁-C₁₀ alkenyl, an aryl group, an alkylaryl group, a heteroaryl group or an alkylheteroaryl group, optionally substituted with one or more of C₁-C₄ alkoxy, hydroxy, amino, carboxylic acid, ester, amide, halogen, CF₃, CHF₂ or CH₂F groups.

6. The multicomponent crystal according to claim 1, wherein the first active pharmaceutical ingredient is selected from lamotrigine, 4-aminopyridine, cytosine, thymine, 5-fluorocytosine, dihydralazine, endralazine, hydralazine, pipofezine, minaprine, cadralazine or cefozopran.

7. The multicomponent crystal according to claim 1, wherein the second active pharmaceutical ingredient is selected from valproic acid and/or a valproate salt, NSAIDs—including salicylate derivative NSAIDs, p-amino phenol derivative NSAIDs, propionic acid derivative NSAIDs, acetic acid derivative NSAIDs, enolic acid derivative NSAIDs and fenamic acid derivative NSAIDs—non-selective cyclo-oxygenase (cox) inhibitors, selective cyclooxygenase 1 (cox 1) inhibitors, selective cyclooxygenase 2 (cox 2) inhibitors or an antibiotic such as oxacillin, ampicillin, amoxicillin, cephalexin, cephalotin, cephalosporin, p-amino-salicylic acid, ciprofloxacin, enrofloxacin, difloxacin or danofloxacin.

8. The multicomponent crystal according to claim 1, wherein the second active pharmaceutical ingredient is a pharmaceutically acceptable excipient.

9. The multicomponent crystal according to claim 8, wherein the second active pharmaceutical ingredient is benzoic acid.

10. The multicomponent crystal according to claim 1, wherein the molar ratio of the first active pharmaceutical ingredient to the second active pharmaceutical ingredient in the multicomponent crystal is 1:2.

11. The multicomponent crystal according to claim 1, wherein the interaction of the nitrogen-containing heterocycle substituted with an amino group of the first active pharmaceutical ingredient and the carboxylic acid group of the second active pharmaceutical ingredient comprises an R₁² (4) synthon.

12. The multicomponent crystal according to claim 1, wherein the interaction of the nitrogen-containing heterocycle substituted with an amino group of the first active pharmaceutical ingredient and the carboxylic acid group of the second active pharmaceutical ingredient comprises an R₂²(8) synthon.

13. The multicomponent crystal according to claim 1, wherein the multicomponent crystal of this first aspect comprises a neutral form of at least one of the first or second active pharmaceutical ingredients.

14. The multicomponent crystal according to claim 1, comprising an ionic form and a neutral form of the first active pharmaceutical ingredient and an ionic form and a neutral form of the second active pharmaceutical ingredient; and wherein the first active pharmaceutical ingredient and the second active pharmaceutical ingredient are organic compounds.

15. The multicomponent crystal according to claim 1 in the form of a medicament.

16. The multicomponent crystal according to claim 1 in the form of a medicament useful in the treatment of epilepsy.

17. A method of preparing a multicomponent crystal comprising at least two active pharmaceutical ingredients, the method comprising the steps of:
a) providing a first active pharmaceutical ingredient comprising a nitrogen-containing heterocycle substituted with an amino group;
b) providing a second active pharmaceutical ingredient comprising a carboxylic acid;
c) combining the first active pharmaceutical ingredient and the second active pharmaceutical ingredient; and
d) crystallising the combination of the first active pharmaceutical ingredient and the second active pharmaceutical ingredient obtained from step c) to provide the multicomponent crystal.

18. A pharmaceutical composition comprising a therapeutically effective amount of a multicomponent crystal according to claim 1 and a pharmaceutically acceptable excipient.

19. The multicomponent crystal according to claim 1, wherein the first active pharmaceutical ingredient is lamotrigine and wherein the second active pharmaceutical ingredient is valproic acid.
